(19) United States
(12) Patent Application Publication
(10) Pub. No.: US 2006/0293606 A1
(43) Pub. Date: Dec. 28, 2006

(54) Personal authentication using heart sound waveform and/or breathing waveform pattern

(76) Inventor: Seijiro Tomita, Tokyo (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 11/454,133

(22) Filed: Jun. 16, 2006

Related U.S. Application Data
(63) Continuation of application No. PCT/JP03/16185, filed on Dec. 17, 2003.

(51) Int. Cl. A61B 5/08

(52) U.S. Cl. 600/532; 600/525

(57) ABSTRACT

We provide quite a novel personal authentication system using heart sound waveform and/or breathing waveform pattern which promptly judges personal authentication of a person to be authenticated with a very high degree of accuracy. The personal authentication system is comprised of a waveform detection means which detects a heart sound waveform pattern and a means which compares a heart sound waveform pattern detected by the waveform detection means with a previously registered heart sound waveform pattern. If the detected heart sound waveform pattern coincides with the registered heart sound waveform pattern, the system authenticates the person to be identical. In such constitution, the system can check the autonomic heart sound which cannot be controlled by a personal will based on the previously registered heart sound waveform pattern in real time, and performs personal authentication of the person to be authenticated with very high accuracy promptly.

### TECHNICAL FIELD

The present invention relates to a personal authentication using heart sound waveform and/or breathing waveform pattern which autonomously changes. As used herein, the term “heart sound” shall mean a sound which is produced by heartbeat of the heart and repeatedly produced by contraction or relaxation of the cardiac chamber, comprising two sounds being heard per heartbeat cycle, and is originated from a closing sound of the valve. The heart sound is comprised of a first heart sound being low and somewhat longer sound and a second heart sound being somewhat high and short. Further, the first heart sound is produced when the atrioventricular valves (mitral valve and tricuspid valve) close at the beginning of contraction phase of the cardiac chamber. The second heart sound is produced when the arterial valves (aortic valve and pulmonary valve) close immediately after contraction phase of the cardiac chamber. The heart sound relates to an open of the valve and a heartbeat, and is a generic term of “short vibration” produced therefrom. On the other hand, “heartbeat” is referred to as “pitch of the heart.” The heartbeat is measured on a surface electrocardiogram which records electrical activity of the cardiac muscle. The electromyogram which is recorded per heartbeat is classified into main three waveforms (P waveform, QRS waveform and T waveform). The “heartbeat” is differentiated from the “heart sound” according to the present description. (Source: Bates Physical Examination, 7 ed [3, P 282]; Medical Diagnosis [91, P 209]; Lange Clinical Cardiology, 6 ed [6, P 58]; Review of Medical Physiology [95, P 561].

### BACKGROUND OF THE INVENTION

Recently, with the rapid progress of information society, requirement for personal authentication technology has been swiftly increased in the ranges from buildings and rooms to information apparatuses and networks. Authentication is required for various places from a place being accessed by a small group to a place being accessed by the general public. Especially, for an apparatus which unspecified majority uses and personal authentication technology which controls enter and leave, it is a key point to provide a low contact, no discomfort with which a person to be authenticated (hereinafter referred to as “user”) feels from the hygiene standpoint, and an impossibility of counterfeit.

Main personal authentication means that is currently used include those such as an authentication by a license which a user owns, an authentication by a PIN number or a password and a biometric authentication based on biological characteristics such as a fingerprint or an iris.

As used herein, the term “biometrics” shall mean “anthropometry”. Personal authentication thereby means a utilization of various physical and behavioral characteristics including fingerprint, sound spectrogram, iris, face and signature which cannot be lent to others. The biometric authentication has advantages such as not being missed, not being stolen, not being forgotten, and neither being counterfeited nor altered, in comparison with an “authentication by possessions” such as a key or a credit card and an “authentication with knowledge” such as a PIN number. Since the authentication is verified by a part of the body or a behavior, it is not necessary to carry the thing with him/her and to remember the information of verifying identity. Further, another advantage is that there is scarcely anxiety that primary information such as physical characteristics may be stolen.

By the way, a heartbeat and electrocardiogram measurement system which picks up electrocardiogram data has been known as a conventional personal authentication method using biometrics. With this system, if an electrode plate separates from the body, measurement is not performed. Further, since the electrode plate has to be attached to the body of a subject for 24 hours, freedom of action of the subject is restricted. The restriction demands on the subject psychologically. To pick up data on the true physical reaction of the subject during 24 hours (in a condition that a subject does not receive any psychological burden; hereinafter, the same condition is applied to) in real time is impossible in the proper sense. This method lacks in versatility, which is an issue to be solved.

As above, conventional personal authentication technologies have a lot of problems, which have not been solved yet. In addition, various systems using conventional personal authentication technologies as above are constituted as a system which does not carry out the same personal authentication again after personal authentication is once verified.

Therefore, with the conventional personal authentication system, it is possible for anyone else to continue using the system by masquerading as the user. For example, after a password is put in at computer startup and then it is authenticated, no means is currently available for preventing anyone else other than the user from using the computer.

In such a system with one-time authentication, even with an authentication system using the above biometrics which is said to be most excellent, if authentication is once performed, even when anyone else other than the user uses the system, there is currently no means provided for preventing abuse at all. There is no stonewalling available for rampant fraud such as a password or fingerprint copying.

The present invention has been accomplished under these circumstances. A primary object of this invention is to provide a personal authentication system which has not been researched before and enables to promptly verify personal authentication with a high degree of accuracy. The system comprises: detecting a heart sound waveform pattern and/or a breathing waveform pattern which is autonomic rhythm that cannot be controlled by personal will in real time based on the heart sound and/or breathing of a user; and verifying the detected with the previously registered heart sound waveform and/or breathing waveform pattern of the user.

Further in this invention, if the user does not keep touching a personal authentication device using heart sound waveform and/or breathing waveform pattern, the apparatus cannot be used. If anyone else touches the apparatus (when the apparatus recognizes a different heart sound and/or breathing from the registered heart sound waveform and/or breathing waveform pattern), the system can take measures such as an apparatus action stop by regarding the access as an unauthorized use. The object is to provide a breakthrough personal authentication system using heart sound waveform and/or breathing waveform pattern which surely prevents an unauthorized use of apparatus.

### DISCLOSURE OF INVENTION

To accomplish the above purposes, personal authentication system using heart sound waveform and/or breathing waveform pattern according to the present invention is characterized as follows.

In a first aspect of the present invention, a personal authentication system comprises: a waveform detection means which detects a heart sound waveform and/or breathing waveform pattern; and a means comparing the heart sound waveform and/or breathing waveform pattern detected by the waveform detection means with the previously registered heart sound waveform and/or breathing waveform pattern to judge, wherein, if the detected heart sound waveform and/or breathing waveform pattern coincides with the registered heart sound waveform and/or the registered breathing waveform pattern, the user is identified.

In a second aspect of the present invention, technically based the personal authentication system using heart sound waveform and/or breathing waveform pattern according to the first aspect, the waveform detection means comprises an air pad being filled inside with a foamed resin and the air and a piezoelectric sensor detecting a change in air pressure in the air pad, wherein the air pressure of the air pad side of the piezoelectric sensor is held and other side thereof is open up to the air so as to form a difference in pressure between the air-filled chamber and the open-up-to-the-air side.

In a third aspect of the present invention, technically based on the personal authentication system using heart sound waveform and/or breathing waveform pattern according to one of the first and the second aspects, other side of the body contacting surface of the air pad is characterized by that a plate material is installed.

In a fourth aspect of the present invention, technically based on the personal authentication system using heart sound waveform and/or breathing waveform pattern according to the third aspect, the plate material is characterized by being formed in a wedge shape where a thickness decreases in one direction.

In a fifth aspect of the present invention, technically based on the personal authentication system using heart sound waveform and/or breathing waveform pattern according to any one of the aspects first to fourth, the waveform detection means is characterized by being installed in parallel.

In a sixth aspect of the present invention, technically based on the personal authentication system using heart sound waveform and/or breathing waveform pattern according to any one of the aspects first to fifth, detection of heart sound waveform and/or breathing waveform with the waveform detection means is characterized by being carried out continuously or more than once.

In a seventh aspect of the present invention, technically based on the personal authentication system using heart sound waveform and/or breathing waveform pattern according to any one of the aspects first to sixth, the personal authentication device is characterized by being installed in a computer mouse or other input means.

In an eighth aspect of the present invention, technically based on the personal authentication system using heart sound waveform and/or breathing waveform pattern according to any one of the aspects first to sixth, the personal authentication device is installed in a body contacting region of a mobile telephone, a portable terminal, a copy machine, a fax machine, a printer, a lighting fixture, a doorknob for building, an electric train, an automobile car, a large size construction machine, a cultivator, an aircraft, a ship and vessel, a bicycle, a two-wheeled motor vehicle, a wireless remote control and an electric appliance.

In a ninth aspect of the present invention, technically based on the personal authentication system using heart sound waveform and/or breathing waveform pattern according to any one of the aspects first to eighth, the personal authentication is characterized by being performed by a combination of the heart sound waveform and/or breathing waveform pattern with any one of personal authentications such as a PIN number authentication, a password authentication, a biometric authentication with fingerprint, iris, face, signature and finger vein pattern and a heartbeat pattern by electrocardiogram.

In a tenth aspect of the present invention, technically based on the personal authentication system using heart sound waveform and/or breathing waveform pattern according to any one of the aspects first to ninth, tension or degree of excitement of a user is characterized by being measured by the heart sound waveform and/or breathing waveform pattern pitch of the user measured by the personal authentication device.

In an eleventh aspect of the present invention, technically based on the personal authentication system using heart sound waveform and/or breathing waveform pattern according to any one of the aspects first to tenth, the personal authentication device registers a heart sound waveform and/or breathing waveform pattern of a user detected by a waveform detector as data, and is equipped with a learning function means for analyzing the recorded data to improve the authentication accuracy and the speed.

### BRIEF DESCRIPTION OF THE DRAWINGS

FIG. 1 is a plan view with a partial section view showing a constitution example of sensor portion of the waveform detector which is used for realizing the personal authentication system using heart sound waveform pattern pattern of the present invention.

FIG. 2 is a cross-section view of the heart sound detection sensor along the line A-A shown in FIG. 1.

FIG. 3 is a cross-section view of the heart sound detection sensor along the line B-B shown in FIG. 1.

FIG. 4 is a block diagram showing a constitution example of the waveform detector.

FIG. 5 is a waveform diagram showing a heart sound waveform.

FIG. 6 is a detection example of the breathing waveform and the heart sound waveform.

FIG. 7 is a block diagram showing a basic constitution of the waveform detector.

FIG. 8 is a cross-section view in the case that the waveform detector is installed in a computer mouse.

### DETAILED DESCRIPTION OF THE PREFERRED EMBODIMENTS

Now, embodiments of the present invention will be described by referring to the accompanying drawings.

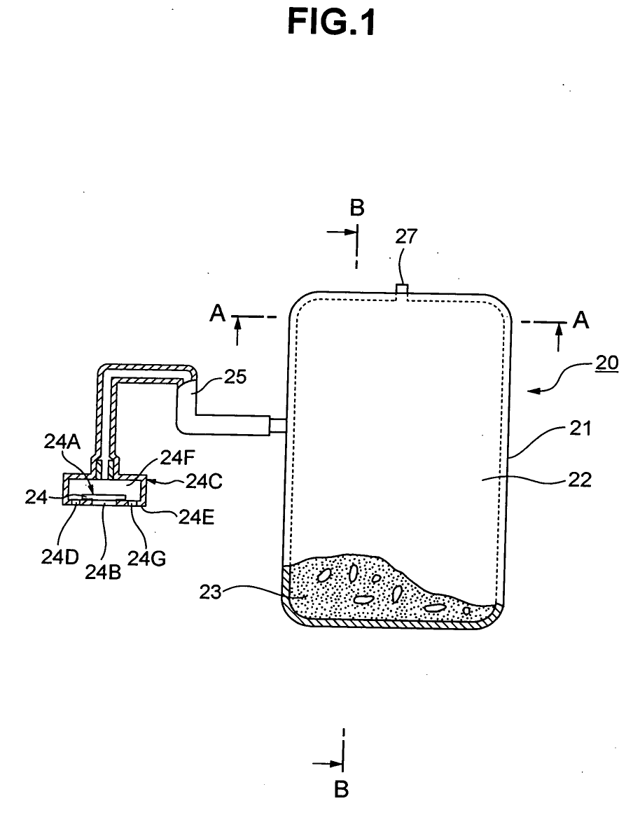

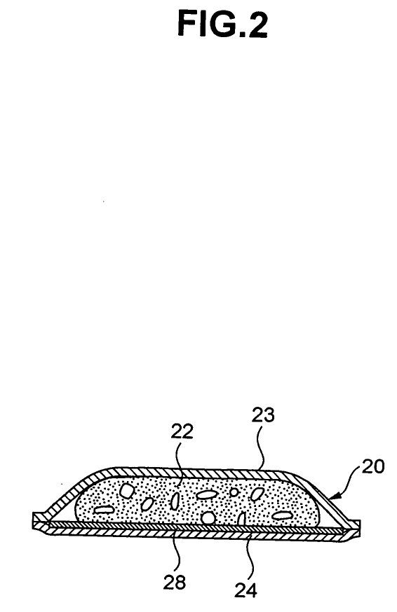

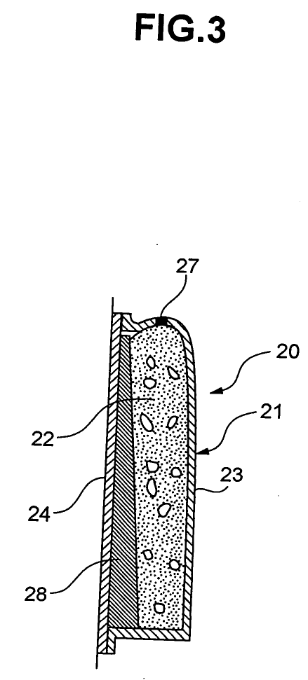

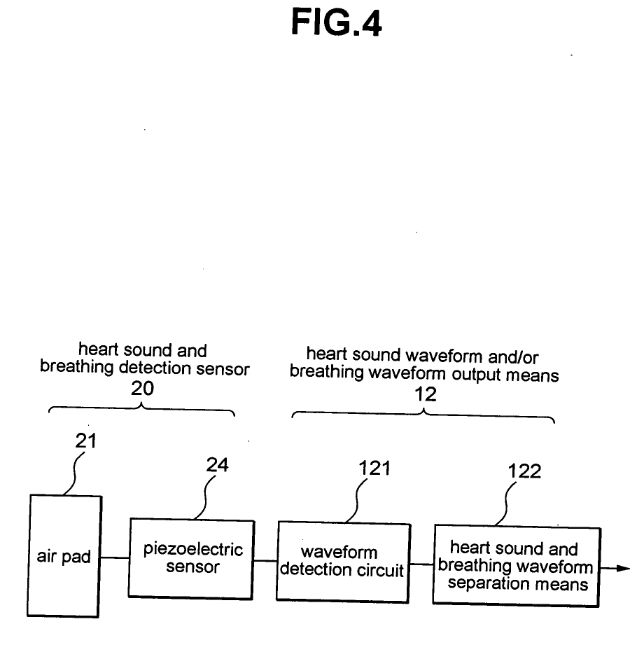

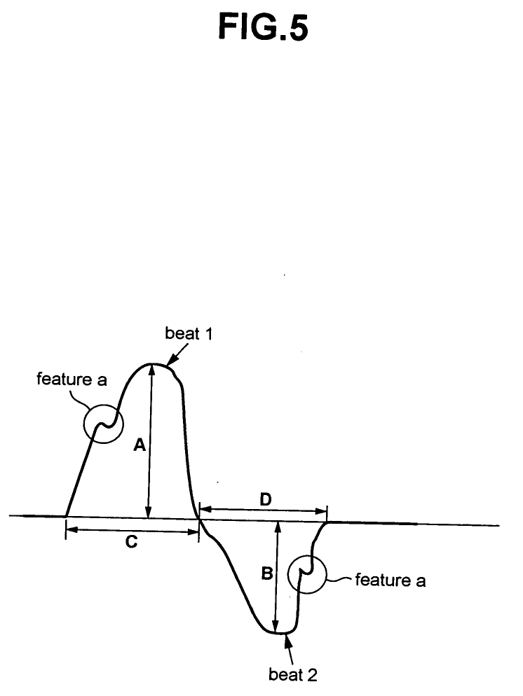

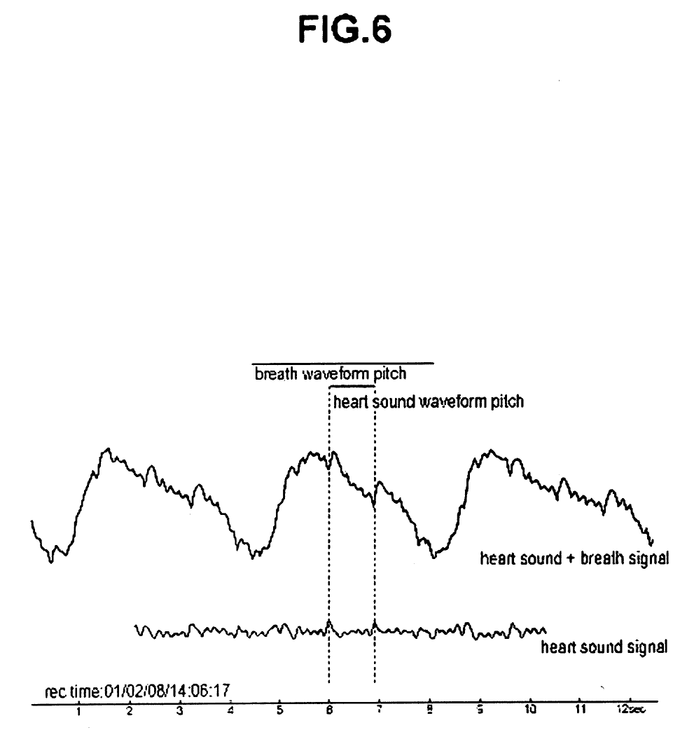

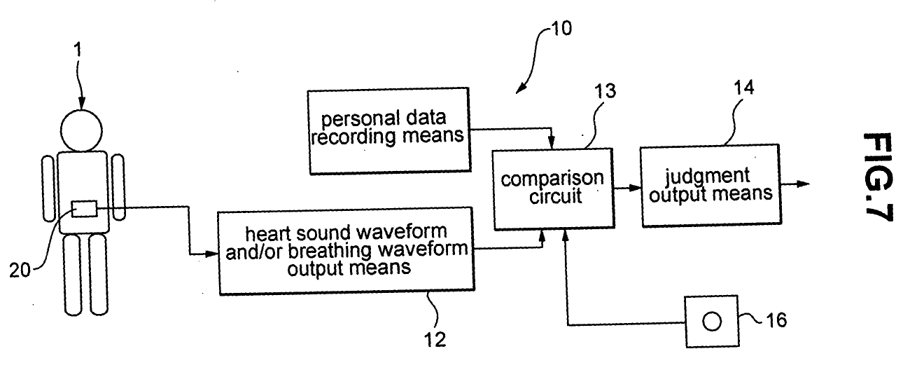

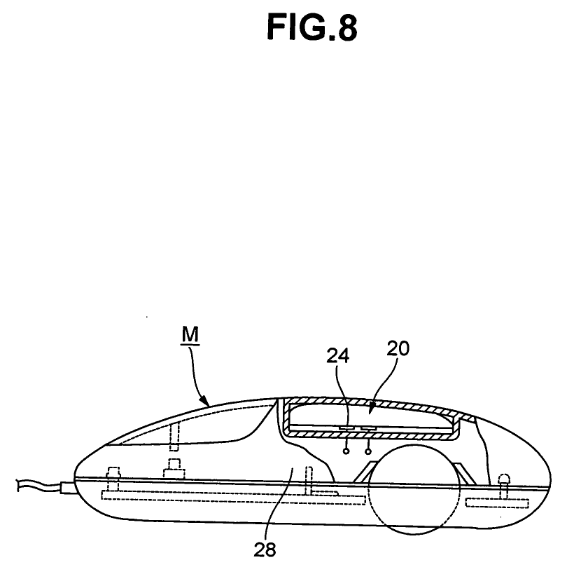

FIGS. 1 to 12 show an embodiment of the waveform detector which is used for realizing a personal authentication system using heart sound waveform and/or breathing waveform pattern of the invention. FIG. 1 represents a plan view with a partial section view showing a constitution example of sensor portion of the waveform detector which is used for realizing a personal authentication system using heart sound waveform pattern of the present invention. FIG. 2 represents a cross-section view seen from the line A-A of the heart sound detection sensor shown in FIG. 1. FIG. 3 represents a cross-section view seen from the line B-B of the heart sound detection sensor shown in FIG. 1. FIG. 4 represents a block diagram showing a constitution example of the waveform detector. FIG. 5 represents a waveform diagram showing a heart sound waveform. FIG. 6 represents a detection example of a breathing waveform and a heart sound waveform. FIG. 7 represents a block diagram showing a basic constitution of the waveform detector. FIG. 8 represents a cross-section view in the case that the waveform detector is installed in a computer mouse.

The waveform detector of the present embodiment comprises a heart sound and breathing detection sensor 20 which is installed in a region where a part of body of the user 1 such as a finger or a hand contacts as a subject of personal authentication. The heart sound and breathing detection sensor 20 comprises an air pad 21 in which is filled inside as a foam resin with a foam urethane resin 23 such as air containing sponge, a piezoelectric sensor 24 which detects a change in air pressure in the air pad 21, a pipe 25 which has flexibility of keeping the inside of the piezoelectric sensor 24 and the air pad 21 air-tight but continuously connecting the interspace therebetween, and a piezoelectric generating device as a power source (not shown).

The foam urethane resin 23 is filled in the air pad 21 for keeping the inside of the air pad 21 at a certain number of dilation. After the air pad 21 has been deformed by an external force, when the external force stops giving an influence, the foam urethane resin 23 tries to restore to the original dilation condition promptly.

The air pad 21 is constituted of a rectangle-shaped bursiform which comprises two plane-shaped epidermides 22 and 23. In order to keep the inside airtight, peripheral portions of the both epidermides 22 and 23 are pasted with an additive which maintains airtightness. It is preferable to use, for example, a rubber or a soft synthetic resin with excellent airtightness for the epidermides 22 and 23 of the air pad 21. In order to enhance the airtightness stronger, it is preferable to form the surfaces with a plurality of layers.

Further, inside the air pad 21, a hard plate material 28 is installed as shown in FIGS. 2 and 3. The plate material 28 is installed in a wedge shape where a thickness decreases in one direction. Therefore, when the air pad 21 is installed, for example, between the wear and the body, since a movable side (epidermides side) of the air pad 21 is attached to the body side, a change in the air pressure inside the air pad 21 accompanied by a change in microseism and by breathing can be surely picked up, the sensitivity of the waveform detector 1 can be kept well. In the case that the air pad 21 is attached to the body by inserting in a pocket of the wear, even if the movable side of the air pad 21 is attached to a contour body, since the movable side is naturally pressed to the body direction by the wedge-shaped plate material 28 to firmly attach to the body, a detection sensitivity of the heart sound waveform and/or breathing waveform pattern can be improved. Further, in the present embodiment, as shown in FIG. 1, since an air pad part 24A of piezoelectric sensor 24 is kept airtight and other part 24B is constituted so as to open up to the air, the system is constituted so as to produce a difference in pressure between the air-filled space side and the air side; and since the system can transmit to the piezoelectric sensor 24 a subtle change in pressure by the heart sound vibration or the breathing vibration which are applied to the air pad 21, a heart sound waveform and/or breathing waveform data can be extracted with a high degree of accuracy. Besides, in the embodiment, an example is explained which the air pad 24A of the piezoelectric sensor 24 is constituted so as to keep airtight, but this invention does not limit to it. A fine pore 24D is formed so as to continuously pass through the air-filled space side of the sensor portion 24C which the piezoelectric sensor 24 is equipped with and the air side, and the air in the air-filled space side is constituted so as to pass through in a compressed condition. In this case, a foam urethane 23 pressed and contracted is prevented from restoring the original shape in the airtight condition and can be returned to the original shape smoothly.

In this embodiment, in the air pad 21, a flow channel 27 which an air pump for filling up the air can be continuously connected is equipped with; and in the flow channel 27, a check valve which prevents the air inside the air pad 21 from flowing out (not shown) is installed. Even if the air pad 21 and the piezoelectric sensor 24 are connected to each other in an airtight condition, since the pressure inside the air pad 21 decreases due to the time-lapse use, the check valve is constituted so as to increase the pressure to a certain value by the air pump. When the pressure inside the air pad 21 reaches a certain value by the air pump, it is desirable, for example, to constitute not to increase the pressure by the check valve any more.

As the above piezoelectric sensor 24, a known piezoelectric pressure sensor may be used for detecting a change in the air pressure inside the air pad 21.

In this embodiment, as above, the piezoelectric sensor 24 is installed in a room 24F of the sensor portion 24C which is continuously connected to a distal end of the pipe 25 and blocks with an adhesive a pore portion 24G which is installed on a bottom portion 24E forming the above chamber by the piezoelectric sensor 24.

A room 24 F which is formed in the air pad 24A of the piezoelectric sensor 24 is constituted so as to keep the air pressure. Besides, other side of the piezoelectric sensor 24 is continuously connected to the air side via the pore portion 24G. The piezoelectric sensor 24 is constituted so that a difference in pressure is produced between the air filling space side and the air side. Since the piezoelectric sensor 24 is constituted in such a way that a difference in pressure can be produced between the air filling space side and the air side of the piezoelectric sensor 24, a heart sound vibration and a breathing vibration applied to the air pad 21 can be surely caught by the piezoelectric sensor 24.

We go on to spell out a heart sound waveform and/or breathing waveform output means 12 which outputs a signal from the heart sound and breathing detection sensor 20 as a heart sound waveform and/or breathing waveform pattern.

FIG. 4 shows a schematic constitution of the output means 12. The output means 12 is constituted of a waveform detection circuit 121 and a heart sound and breathing waveform separation means 122. The waveform detection circuit 121 detects a heart sound and breathing waveform detected by the heart sound and breathing detection sensor 20 which comprises the air pad 21 and the piezoelectric sensor 24. The heart sound and breathing waveform separation means 122 separates a heart sound waveform and a breathing waveform.

The heart sound and breathing waveform separation circuit 122 is connected to a comparison circuit 13 which is described later, and is constituted so as to prevent a malfunction and an erroneous judgment (For algorithm for separating heart sound waveform and breathing waveform, refer to PCT Application No. PCT/03/05711). The heart sound and breathing waveform separation circuit 122 may be constituted so that the detected heart sound waveform and breathing waveform data can be transmitted via a self-contained communication circuit (not shown). In the present invention, there provide constitutional possibilities: only heart sound waveform which is detected by the heart sound and breathing detection sensor 20 and then separated is used as an individuation authentication means; only breathing waveform which is detected by the heart sound and breathing detection sensor 20 and then separated is used as a personal authentication means; and both waveforms which are detected by the heart sound and breathing detection sensor 20 and then separated are used as a personal authentication means.

The above heart sound waveform or the breathing waveform according to the present invention can be compared by a known general comparison method. For example, as shown in FIG. 5, with using the heart sound waveform or breathing waveform comparison method a heart sound comprising a first sound and a second sound is compared on a vibration ratio (A and B) between the first sound and the second sound, or a comparison of the durations allows to check whether a first sound and a second have a same waveform. Special feature ‘a’ which is involved in the first sound and the second sound may be picked up for comparison. Further, using FFT (Fast Fourier Transform), a comparison may be performed on data whose frequency components contained in the heart sound are transformed on the time-basis. Besides, there is a method of detecting relaxed cyclic fluctuation (circadian rhythm) from variation in the heart sound.

The heart sound waveform signal and the breathing waveform signal obtained in such a way is transmitted to the comparison circuit 13 shown in FIG. 6 and then transmitted further to a judgment output means 14. The code 16 in FIG. 6 shows an information storage means. As the information storage means 16, known and optional matter such as a magnetic tape, a magnetic disk, an optical disk, a solid-state memory, an IC chip and a micro chip can be used.

The comparison circuit 13 compares a heart sound waveform and/or breathing waveform detected from the output means 12 with a heart sound waveform and/or breathing waveform pattern of the user which is previously registered in the information storage means 16. The judged results are transmitted to the judgment output means 14.

The judgment output means 14 transmits to a device requiring the personal information the information where a heart sound waveform and/or breathing waveform detected from the output means 12 coincides or does not coincide with the heart sound waveform and/or breathing waveform pattern of the user which is previously registered in the information storage means 16. Relevant devices perform necessary treatment based on the respective judgment to finish a personal authentication work.

In this manner, if a heart sound waveform and/or breathing waveform pattern of the user is registered in various devices, apparatuses or information storage means of various systems, when requiring personal authentication, a heart sound waveform and/or breathing waveform detected in real time can be compared with the registered heart sound waveform and/or breathing waveform pattern of the user for check. Thus, this method enables to judge personal authentication with a very degree of accuracy even in a simple system and then to take necessary action.

As shown FIG. 9, for example, the waveform detector equipped with the heart sound and breathing detection sensor 20 of such constitution is mounted in a mouse M which is connected to a computer. When a heart sound waveform and/or breathing waveform detected by the heart sound and breathing detection sensor 20 that is mounted in the mouse M is compared with the heart sound waveform and/or breathing waveform pattern of the user which is registered in the computer body (not shown), the computer can be constituted in the following way: if these coincide with each other, the computer may be used; if these do not coincide with each other, the computer is automatically shut down. Further, if the computer is rebooted in a specific manner, the computer can be constituted so as not to be available. The computer can be constituted so that personal authentication is continuously performed and without keeping a finger or a hand of the user on the mouse M the computer is not available for continuous operation. If a different heart sound and/or breathing is detected during use of the computer, the computer can be constituted so as to automatically log ff immediately and then to prohibit a continuous application thereafter. In this case, the term “continuous application” does not mean a continuity that a finger or a hand of the user has to be kept touching to the mouse M, and does include a continuity that after the user once touched the mouse M and takes off the finger or the hand, if a user who touched the mouse M is the user, the computer is available for operation.

The installed region of the heart sound and breathing detection sensor 20 is not limited to the mouse M. Installation in other input means, for example, a key button of keyboard, a region where a palm of the hand contacts the computer, or a tablet for computer can also produce a similar effect.

Further in this invention, the system is constituted so that the personal authentication process is performed more than once during a period when the body of the user is contacting to the heart sound and breathing detection sensor 20; and if a heart sound waveform and/or breathing waveform pattern is not continuously detected and personal authentication is not continuously repeated more than once, the user is not authenticated as the same user and then measures against abuse such as prohibition of using the computer may be taken.

Personal authentication by the system may be constituted in a manner so that the heart sound and breathing detection sensor 20 continuously detects the heart sound waveform and/or breathing waveform more than once and the detected heart sound waveform and/or breathing waveform pattern is compared with the registered heart sound waveform and/or breathing waveform pattern more than once at optional timing. In this case, since the timing of personal authentication is optional and cannot be forecasted, the abuse can be more accurately prevented. As used herein, the term ‘optional’ means all timings except for ‘at regular time intervals’, and include a continuation of personal authentication at irregular timing and a continuation of personal authentication at random timing.

In this embodiment, the authentication work may be constituted so that a heart sound waveform and/or breathing waveform that the heart sound and/or breathing detection sensor 20 continuously detects is compared with the registered heart sound waveform and/or breathing waveform pattern at regular time intervals (for example, per pulse interval) more than once for leading a judgment.

As explained above, the personal authentication system using heart sound waveform and/or breathing waveform pattern of this embodiment is constituted so that a user is authenticated to be identical when a heart sound waveform and/or breathing waveform detected by the heart sound and/or breathing detection sensor 20 coincides with the registered heart sound waveform and/or breathing waveform pattern. Further, a heart sound produced by the heart pulse which is autonomic that cannot be controlled by will and a breathing which is not completely autonomic are used. Besides, since different person has a different heart sound waveform and/or breathing waveform pattern, a comparison and judgment ensures personal authentication, and authentication by the heart sound waveform and/or breathing waveform pattern requires neither taking a thing with the user nor remembering information for authenticating the user. Advantages such as no anxiety to be stolen, not being lost, not to be forgotten, not to be counterfeited or altered are obtained.

Even after a user is authenticated to be the user, since various apparatuses, devices or systems repeat approval or disapproval of continuous use thereof, these abuses are prevented at far higher level than a conventional personal authentication.

Further, in this invention, every heart sound waveform and/or breathing waveform of the user detected by the heart sound and/or breathing detection sensor 20 is recorded and stored as data. With applying a learning function means which analyzes the recorded data, even if the heart sound waveform and/or breathing waveform pattern changes according to the change of health condition and physical condition of the user, this pattern is automatically updated. Even if the heart sound waveform and/or breathing waveform gradually changes according to the aging, the system can accurately judge the user without modifying the authentication system. Besides, the authentication speed can be improved. With using the system, a health of employee and the family can be controlled without being known to the person.

In the above embodiment, an explanation is made of an example which the heart sound and/or breathing detection sensor 20 is installed in the mouse M and so forth, but the embodiment does not limit the scope of the present invention. For example, the system can be installed in a body contacting region of a mobile telephone, a portable terminal, a copy machine, a fax machine, a printer, a lighting fixture and an electric appliance.

Since the heart sound and/or breathing detection sensor 20 is installed in a doorknob of building, only when the user grasps the doorknob and is authenticated to be the user (whose heart sound waveform and/or breathing waveform pattern is registered), the doorknob can be constituted so as to move in an opening direction or the lock device is constituted so as to open the key.

The heart sound and/or breathing detection sensor 20 may be installed in a body contacting region of an electric train, an automobile car, a large size construction machine, a cultivator, an aircraft, a ship and vessel, a bicycle, a gear shaft of two-wheeled motor vehicle, a handle and so forth, in which only user (whose heart sound waveform and/or breathing waveform pattern is registered) can operate the vehicles and so forth. When a heart sound except for the registered heart sound waveform and/or breathing waveform pattern is detected, the sensor may be constituted so that a power source of the vehicles and so forth is automatically put off and cannot be restarted without using a specific method or so that a handle and so forth is locked. Antitheft or abuse of the vehicles and so forth may be surely protected.

Further, the heart sound and/or breathing detection sensor 20 may be installed in a wireless remote control for a television, an air conditioning machine or a vehicle door lock and open. Even if a third party other than the user touches an operation key, only user whose heart sound waveform and/or breathing waveform is detected to be identical with the registered heart sound waveform and/or breathing waveform pattern can normally send a signal. If a different heart sound and/or breathing is detected, a control signal is not sent from the operation key. If other steals the wireless remote control and then uses, operation of apparatuses, devices and systems cannot be controlled. These abuses can be surely prevented.

In the above embodiment, explanation is made of an example in which personal authentication using heart sound waveform and/or breathing waveform pattern is performed by “heart waveform and/or breathing waveform pattern” in biometrics, but the embodiment does not limit the scope of the present invention. In combination with a personal authentication system using biometrics based on the biological specific features which uses one of authentications by a conventional PIN number, personal authentication by a password, a fingerprint, a voice pattern, an iris, a face, a signature, a finger vein pattern and an electro cardiogram waveform data by electro cardiogram, a hybrid with the personal authentication system is performed without abolishing an existing personal authentication system. In this case, since infrastructure of the existing system can be utilized, the personal authentication system can be used for multipurpose with a small equipment investment.

In this invention, when the user is so nervous or excited that the heart sound waveform and/or breathing waveform pattern pitch of the user which is detected by the waveform detector 10 is different from the registered heart sound waveform and/or breathing waveform pattern pitch, since the heart sound waveform and/or breathing waveform pattern of the user becomes shorter than the heart sound waveform and/or breathing waveform pattern at cool condition, the system can be constituted so that the personal authentication work is suspended for a while or aborted and the report is informed the user by a caution or a warning beep, and then personal authentication may be performed from the first step, or by informing a third party such as the police or a contracted security company of the situation, an abuse due to threat and so forth may be protected before happens.

### INDUSTRIAL APPLICABILITY

As described above, a personal authentication system using heart sound waveform and/or breathing waveform pattern according to the present invention has the following advantages.

According to a first aspect of this invention, a personal authentication system comprises: a waveform detection means which detects a heart sound waveform pattern and/or a breathing waveform pattern; and a means which compares the heart sound waveform pattern and/or the breathing waveform pattern that the waveform detection means detects with the previously registered heart sound waveform pattern and/or the breathing waveform pattern and then judges. The personal authentication system is constituted in a manner so that if the detected waveform pattern coincides with the registered waveform pattern the user is authenticated to be identical. In combination with an autonomic heart waveform which cannot be controlled by a personal will, a breathing waveform or a combination thereof, a heart sound waveform and/or a breathing waveform can be easily checked in real time based on the previously registered heart sound waveform and/or the breathing waveform of the authenticated user, and personal authentication of the authenticated user is promptly judged with a very high degree of precision.

According to a second aspect of this invention, the waveform detection means comprises: an air pad which is filled inside with foam resin and the air; and a piezoelectric sensor which detects a change in air pressure in the air pad. Placing the air pad, for example, between the wear and the body, a change in the heart sound (vibration due to the pulse interval of the heart) can be surely detected. Since an air pad side of the piezoelectric sensor is kept airtight and other side is formed so as to open up to the air to produce a difference in pressure between the air-filled space side and the open-up-to-the-air side, and since the system can transmit to the piezoelectric element a subtle change in pressure by the heart sound waveform and/or breathing waveform pattern which is applied to the air pad, a heart sound waveform and/or breathing waveform data can be extracted with a high degree of accuracy. Furthermore, since the detection can be performed without direct contact to the skin or exposure of the skin, the above detection work can be easily performed without holding the body of the authenticated user.

According to a third aspect of the present invention, on an opposite side of the body contacting surface of the air pad, a plate material is installed. A portion of measuring a change in pressure due to a heart sound waveform and/or breathing is only movable part. Since the plate portion is uninvolved in the measurement, a change in a continued heart sound or breathing can be surely detected with a high degree of sensitivity.

According to a fourth aspect of the present invention, since the plate material is formed in a wedge shape where a thickness thereof decreased in one direction, if a movable side of the air pad contacts to any region of the body, the movable side is naturally pressed in the body direction by the wedge-shaped plate material to attach to the body and the inside air pressure is uniformized. Therefore, a subtle change in the heart sound waveform and breathing can be surely detected with high sensitivity.

According to a fifth aspect of the present invention, since a waveform detection means is installed in parallel, if comparing information from the both waveform detection means, it is easy, for example, to make specific features of the heart sound waveform and breathing form apparent. Further, when making an antiphase or making the waveform gurge in opposite phase, an external sound can be cancelled and clearer analysis can be conducted promptly.

According to a sixth aspect of the invention, since a detection of a heart sound waveform and/or breathing waveform by the waveform detection means is constituted so as to perform continuously or more than once, authentication of the identity is carried out per personal authentication which is conducted continuously or more than once at optional timing. In the case that a heart sound waveform and/or breathing waveform pattern other than the registered heart sound waveform and/or breathing waveform pattern of the user is detected, the devices requiring personal authentication can be immediately stopped to surely prevent an abuse. As used herein, the term ‘optional’ means all timings except for ‘at regular time intervals’, and includes a continuation of personal authentication at irregular timing and a continuation of personal authentication at random timing.

According to a seventh aspect of the present invention, since the personal authentication system is installed in a computer mouse and other input means, the system can be constituted so as not to be used unless the user who is previously registered touches the input means such as a mouse or keeps touching the mouse. The system can be also constituted in a manner that, if personal authentication is performed per program run step or program process step, a user other than the user cannot use the computer continuously. Further, for example, a heart sound waveform pattern of the user can be sent attached to the file name at each shipment of e-mail. If a heart sound waveform pattern of the shipper is previously registered in an address book of a receiver, the received mail can be confirmed whether it is really made by the user. This effect can be obtained through a continuous authentication work.

According to an eighth aspect of the present invention, the personal authentication system is installed in a body contacting region of a mobile telephone, a portable terminal, a copy machine, a fax machine, a printer, a lighting fixture, a doorknob for building, an electric train, an automobile car, a large size construction machine, a cultivator, an aircraft, a ship and vessel, a bicycle, a two-wheeled motor vehicle, a wireless remote control and an electric appliance. Thus, only the user with the registered heart sound waveform pattern can use these equipment. In the case that a different heart sound is detected, operation of these equipment can be promptly stopped. The system improves the security dramatically.

To be more precise, in the case of a mobile telephone, a portable terminal, a copy machine, a fax machine and a printer, personal authentication is performed at startup of the power supply or at a time of keeping in contact with an optional portion or a key of the main body. If the present heart sound waveform and/or breathing waveform coincides with the registered heart sound waveform and/or breathing waveform pattern of the mobile telephone, the portable terminal, the copy machine, the fax machine and the printer, the mobile telephone, the portable terminal, the copy machine, the fax machine and the printer may be used. If not coinciding, the power supply of the mobile telephone, the portable terminal, the copy machine, the fax machine and the printer turn off and cannot be re-started up without using a specific method. Hence, abuse of the mobile telephone, the portable terminal, the copy machine, the fax machine and the printer can be surely prevented. Furthermore, alteration or copy of data stored in the mobile telephone, the portable terminal, the copy machine, the fax machine and the printer may be prevented. In the case of the lighting fixture and the electric appliance, when using these, a heart sound waveform and/or breathing waveform is detected by touching a finger to the personal authentication system and then the detected heart sound waveform and/or breathing waveform is checked out against the registered heart sound waveform and/or breathing waveform pattern. If the both parties coincide with each other, the apparatuses are available. If the both parties do not coincide with each other, the power supply turns off. In this manner, unauthorized use of the apparatuses can be surely prevented.

Moreover, in the case that the personal authentication system is installed in a doorknob, only when the user holds the doorknob and is authenticated to be the user (a user whose heart sound waveform and/or breathing waveform pattern is registered), the doorknob can be movable in the direction to open the door or the lock system can be unlocked. Since conventional lock open keys can be abolished, there will be no trouble such that due to loss of key a user cannot enter into building or room. Further, since a keyhole can be eliminated, a lock open key by picking can be completely swept away to considerably improve security. Even if a user other than the user holds a doorknob, the door does not move or a door lock system does not act and surely prevents a user other than the user from entering into the building or the room.

In the case that the personal authentication system is installed in the body contacting region of an electric train, an automobile car, a large size construction machine, a cultivator, an aircraft, a ship and vessel, a bicycle, a gear shaft of the two-wheeled motor vehicle, a handle and so forth, only the user (a user whose heart sound waveform and/or breathing waveform pattern is registered) can operate the vehicles and so forth. In the case that a heart sound other than the registered heart sound waveform and/or breathing waveform pattern is detected, the power supply of the vehicles and so forth automatically turns on and cannot be re-started up without a specific method or the handle and so forth can be locked to surely prevent a theft and abuse of the vehicles and so forth.

What is more, in the case that the personal authentication system is installed in a wireless remote control, even if a third party other than the user touches an operation key, an authorized user whose heart sound is detected to be identical with the registered heart sound waveform and/or breathing waveform pattern can normally transmit a signal. If a different heart sound is detected, a control signal is not transmitted from the operation key. Even if anyone else steals and uses the remote control, he/she cannot control actuation of the various apparatuses, devices and systems. Thus, these abuses can be surely prevented.

According to a ninth aspect of the present invention, personal authentication is constituted in a combination of authentication by a heart sound waveform and/or breathing waveform pattern according to any one of the aspects first to eighth with authentication by a pin number, an authentication by a password, an authentication by a fingerprint, a voiceprint, an iris, a face, a signature and a finger vein pattern using biometrics, and a heartbeat pattern by electrocardiogram. Thus, accuracy of security can be dramatically improved and a hybrid with a personal authentication system is performed without abolishing existing personal authentication system. In this case, since an infrastructure of existing system can be used, it is possible to install a personal authentication system of the present invention with small equipment investment.

According to a tenth aspect of the present invention, with a heart sound waveform and/or breathing waveform pattern pitch of a user detected by the personal authentication system, a system is constituted so that tension or excitement of the user can be measured. When the detected heart sound and/or breathing pitch is different from the registered heart sound waveform and/or breathing waveform pattern pitch, even if the detected is a heart sound and/or breathing of the user, the detected waveform pattern pitch becomes shorter than the registered heart sound waveform and/or breathing waveform pattern pitch at a time of keeping cool. Then, the personal authentication work is suspended or stopped, the report is informed the user by a caution or a warning beep in order to let the user do personal authentication from the first step. Alternatively, informing a third party such as the police or a contracted security company of the situation, abuse by threat and so forth can be prevented before happens.

According to a twelfth aspect of the invention, a personal authentication system records as data a heart sound waveform and/or breathing waveform pattern of the user detected by a heart sound and/or breathing detector and is equipped with a learning function means analyzing the recorded data. Hence, even if the sound waveform and/or breathing waveform changes due to the health condition or physical condition of the user, the registered data is automatically updated as needed and a personal authentication accuracy and an authentication speed can be improved.

1. A personal authentication system using heart sound waveform and/or breathing waveform pattern, comprising:
a waveform detection means which detects a heart sound waveform pattern and/or a breathing waveform pattern; and
a means which compares the heart sound waveform pattern and/or the breathing waveform pattern that the waveform detection means detects with the previously registered heart sound waveform pattern and/or the registered breathing waveform pattern and then judges,
wherein the waveform detection means comprises an air pad being filled inside with a foamed resin and the air and a piezoelectric sensor detecting a change in air pressure in the air pad,
wherein the air pressure of the air pad side of the piezoelectric sensor is held and other side thereof is open up to the air so as to form a difference in pressure between the air-filled chamber and the open-up-to-the-air side, and
wherein if the detected waveform pattern coincides with the registered waveform pattern the user is identified.

2. (canceled)

3. A personal authentication system using heart sound waveform and/or breathing waveform pattern according to claim 1, wherein a plate material is installed on an opposite side of a body contacting surface of the air pad.

4. A personal authentication system using heart sound waveform and/or breathing waveform pattern according to claim 3, wherein the plate material is formed in a wedge shape where a thickness thereof is decreased in one direction.

5. A personal authentication system using heart sound waveform and/or breathing waveform pattern according to claim 1, wherein the waveform detection means is installed in parallel.

6. A personal authentication system using heart sound waveform and/or breathing waveform pattern according to claim 1, wherein a detection of a heart sound waveform and/or breathing waveform by the waveform detection means is performed continuously or more than once.

7. A personal authentication system using heart sound waveform and/or breathing waveform pattern according to claim 1, wherein the personal authentication system is installed in a computer mouse and other intelligent input means.

8. A personal authentication system using heart sound waveform and/or breathing waveform pattern according to claim 1, wherein the personal authentication device is installed in a body contacting region of a mobile telephone, a portable terminal, a copy machine, a fax machine, a printer, a lighting fixture, a doorknob for building, an electric train, an automobile car, a large size construction machine, a cultivator, an aircraft, a ship and vessel, a bicycle, a two-wheeled motor vehicle, a wireless remote control and an electric appliance.

9. A personal authentication system using heart sound waveform and/or breathing waveform pattern according to claim 1, wherein personal authentication is performed by a combination of an authentication by the heart sound waveform pattern with one of personal authentications such as an authentication by a pin number, an authentication by a password, an authentication by a fingerprint, a voiceprint, an iris, a face, a signature and a finger vein pattern using biometrics, and a heartbeat pattern by electrocardiogram.

10. A personal authentication system using heart sound waveform and/or breathing waveform pattern according to claim 1, wherein tension or excitement of a user can be measured with a heart sound waveform and/or breathing waveform pattern pitch of the user detected by the personal authentication device.

11. A personal authentication system using heart sound waveform and/or breathing waveform pattern according to claim 1, wherein the personal authentication device records as data a heart sound waveform and/or breathing waveform pattern of the user detected by the waveform detector and is equipped with a learning function means analyzing the recorded data for improving authentication accuracy of the user and authentication speed.

12. A personal authentication system using heart sound waveform and/or breathing waveform pattern according to claim 3, wherein the waveform detection means is installed in parallel.

13. A personal authentication system using heart sound waveform and/or breathing waveform pattern according to claim 4, wherein the waveform detection means is installed in parallel.

14. A personal authentication system using heart sound waveform and/or breathing waveform pattern according to claim 3, wherein a detection of a heart sound waveform and/or breathing waveform by the waveform detection means is performed continuously or more than once.

15. A personal authentication system using heart sound waveform and/or breathing waveform pattern according to claim 4, wherein a detection of a heart sound waveform and/or breathing waveform by the waveform detection means is performed continuously or more than once.

16. A personal authentication system using heart sound waveform and/or breathing waveform pattern according to claim 5, wherein a detection of a heart sound waveform and/or breathing waveform by the waveform detection means is performed continuously or more than once.

17. A personal authentication system using heart sound waveform and/or breathing waveform pattern according to claim 3, wherein tension or excitement of a user can be measured with a heart sound waveform and/or breathing waveform pattern pitch of the user detected by the personal authentication device.

18. A personal authentication system using heart sound waveform and/or breathing waveform pattern according to claim 5, wherein tension or excitement of a user can be measured with a heart sound waveform and/or breathing waveform pattern pitch of the user detected by the personal authentication device.

19. A personal authentication system using heart sound waveform and/or breathing waveform pattern according to claim 6, wherein tension or excitement of a user can be measured with a heart sound waveform and/or breathing waveform pattern pitch of the user detected by the personal authentication device.

20. A personal authentication system using heart sound waveform and/or breathing waveform pattern according to claim 3, wherein the personal authentication device records as data a heart sound waveform and/or breathing waveform pattern of the user detected by the waveform detector and is equipped with a learning function means analyzing the recorded data for improving authentication accuracy of the user and authentication speed.

21. A personal authentication system using heart sound waveform and/or breathing waveform pattern according to claim 5, wherein the personal authentication device records as data a heart sound waveform and/or breathing waveform pattern of the user detected by the waveform detector and is equipped with a learning function means analyzing the recorded data for improving authentication accuracy of the user and authentication speed.
